(19) United States
(12) Patent Application Publication
(10) Pub. No.: US 2018/0268109 A1
(43) Pub. Date: Sep. 20, 2018

(54) MEDICAL KIOSK SYSTEM AND METHOD

(71) Applicant: Mayur Ramgir, Boston, MA (US)

(72) Inventor: Mayur Ramgir, Boston, MA (US)

(21) Appl. No.: 15/636,488

(22) Filed: Jun. 28, 2017

Related U.S. Application Data
(60) Provisional application No. 62/471,893, filed on Mar. 15, 2017.

(51) Int. Cl. G06F 19/00; A61B 5/0205

(52) U.S. Cl. CPC G06F 19/3418; G06F 19/322; G01D 5/00; A61B 5/02055; G06F 19/3462

(57) ABSTRACT

A method comprising the steps of providing a first standalone device, configured for capturing details and medical information of the patient. The first standalone device comprising a plurality of sensors and devices. A server in communication with the first standalone device. The first standalone device configured to communicate the medical information to the server. A second standalone device, configured for interacting with a medical practitioner. The server in communication with the second standalone device, configured to communicate the medical information of the patient to the second standalone device. A diagnosis is communicated by the second standalone device to the first standalone device via the server. The first standalone device is configured to provide the patient with a prescription or a medication.

### CROSS-REFERENCE TO RELATED APPLICATIONS

The present Utility patent application claims priority benefit of the U.S. provisional application for patent Ser. No. 62/471,893 entitled “REMOTE DOCTOR CONTROLLED HUMAN VITALITY CHECK/VOICE DIRECTED IN-CAR MOVABLE ROBOTIC ARM IMAGE CAPTURE DEVICE” 15 Mar. 2017 under 35 U.S.C. 119(e). The contents of this related provisional application are incorporated herein by reference for all purposes to the extent that such subject matter is not inconsistent herewith or limiting hereof.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to a medical kiosk. More particularly, certain embodiments of the invention relate to an automated medical kiosk remotely controlled by a medical practitioner.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. Availability of qualified medical practitioners, particularly in remote areas, remains a continuing challenge faced by many countries in the world. One solution includes making medical kiosks available in the remote areas. The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that medical kiosks may be available in remote areas with limited capabilities like video conferencing with doctors.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary system for providing medical kiosk services, in accordance with an embodiment of the present invention;

FIG. 2 illustrates an architecture of an exemplary medical kiosk service system, in accordance with an embodiment of the present invention;

FIG. 3 illustrates an exemplary medical kiosk service system, in accordance with an embodiment of the present invention, in accordance with an embodiment of the present invention;

FIG. 4 illustrates a portion of an exemplary medical kiosk service system, in accordance with an embodiment of the present invention;

FIG. 5 illustrates a portion of an exemplary medical kiosk service system, in accordance with an embodiment of the present invention;

FIG. 6 illustrates a portion of an exemplary medical kiosk service system, in accordance with an embodiment of the present invention;

FIG. 7 illustrates a portion of an exemplary medical kiosk service system;

FIGS. 8A through 8C is a flow chart illustrating an exemplary process for using a medical kiosk, in accordance with an embodiment of the present invention;

FIG. 9 is a block diagram depicting an exemplary client/server system which may be used by an exemplary web-enabled/networked embodiment of the present invention; and

FIG. 10 illustrates a block diagram depicting a conventional client/server communication system.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Embodiments of the invention disclosed herein include in one embodiment, a medical kiosk that may be enabled to collect body vitals, and may include but is not limited to sensors like blood pressure, blood sugar, etc. . . . In one embodiment, the medical kiosk may include remote diagnosis with complete preliminary body vitals data along with blood and urine sample by a medical practitioner, in addition to video conferencing with a medical practitioner. In one embodiment, the medical kiosk may include an automated remotely prescribed medical dispensing unit. Medical practitioners may prescribe medicine remotely and the medical kiosk may dispense the prescribed medicine from its stock. In an embodiment, where the prescribed medicine may not be available in the medical kiosk, the request may be sent to a nearby storage warehouse and may be delivered by a person, a drone, an unmanned vehicle, and the like. In one embodiment, the medical kiosk may print the detailed prescription provided by the medical practitioner. Any analysis that the patient or a medical practitioner may want to view may be provided on a display module of a computer system that may include a designated software program (herein after at times referred to as designated computer program). In one embodiment, the medical kiosk may connect to a cloud database. A patient may thus be enabled to use any medical kiosk from anywhere in the world and get a uniform service and if preferred and available obtain the services of a particular medical practitioner. In certain embodiments, the medical kiosk may include an X-ray machine, such that the medical practitioner may remotely guide a patient to get an X-ray and send it to the medical practitioner. In one embodiment, the X-ray machine may be inbuilt in the medical kiosk In another embodiment, the X-ray machine may be externally connected to the medical kiosk. In one embodiment, the X-ray may be sent to the medical practitioner as soon as it is taken without storing in the medical kiosk and may be stored in the server. In one embodiment, the X-ray may be temporarily stored in the medical kiosk till the X-ray is completed transitioned to the server. In certain embodiments, the medical kiosk may include ultrasound], such that the medical practitioner may remotely guide a patient to use an ultrasound belt and control the probe to take an ultrasound scan and send it to the medical practitioner. In one embodiment, the ultrasound belt may be inbuilt in the medical kiosk In another embodiment, the ultrasound belt may be externally connected to the medical kiosk.

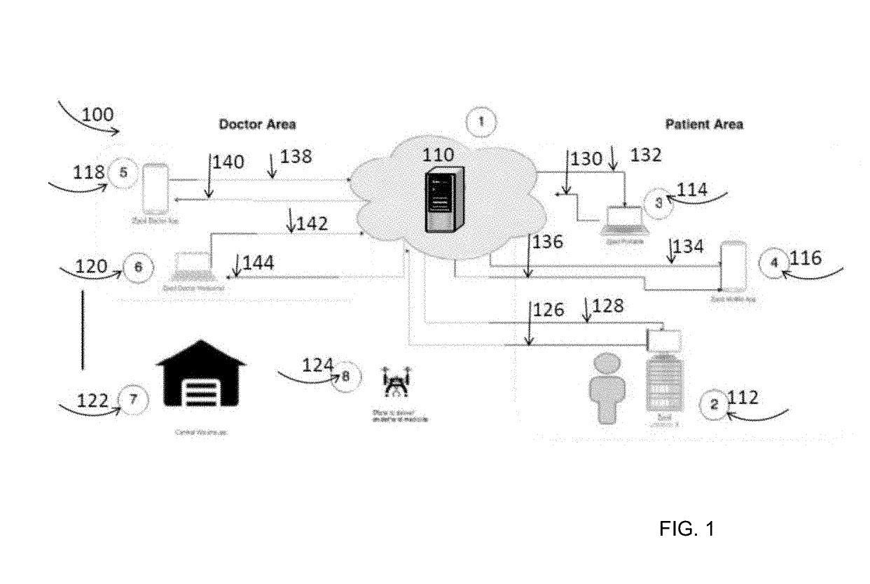

Referring to FIG. 1 is illustrated an exemplary system for providing medical kiosk services, in accordance with an embodiment of the present invention. The system 100 may include a server 110. The system 100 may also include a set of devices for a patient to use and a set of devices for a medical practitioner to use. The set of devices for a patient to use may include a standalone device 112 which may include but is not limited to all sensors; a standalone personal device 114 which may include all sensors; and a standalone mobile device 116 with a designated computer program and an option of being connected to portable sensors. The set of devices for a medical practitioner to use may include but is not limited to a standalone mobile device 118 which enables a medical practitioner to check patient record, prescribe medicine, etc. . . . , and a standalone computer device 120 with a designated software program to enable a medical practitioner to check patient record, prescribe medicine, etc. . . . The system 100 may further include a warehouse to store prescription medication 122, and a medication delivery device 124. In one embodiment, the set of devices for a user/patient to use may be referred to as a first standalone device and the set if devices for a medical practitioner to use may be referred to as a second standalone device.

In one embodiment, the standalone device 112 may be in communication (input information to server 126, output information from server 128) with the server 110. In one embodiment, the standalone personal device 114 may be in communication (input information to server 130, output information from server 132) with the server 110. In one embodiment, the standalone mobile device 116 may be in communication (input information to server 134, output information from server 136) with the server 110.

In one embodiment, the standalone mobile device 118 may be in communication (input information to server 138, output information from server 140) with the server 110. In one embodiment, the standalone computer device 120 may be in communication (input information to server 142, output information from server 144) with the server 110.

During a typical operation, when a patient in a remote location requires the services of a medical practitioner, the patient may make use of the standalone device 112 i.e., an automated medical kiosk. The patient may start the login process with registering themselves using a simple form displayed on a display module of the standalone device 112. In one embodiment display module may include an interactive screen which may allow patients and remote doctors to communicate with each other The patient may also provide fingerprint/retinal details using fingerprint/retina scanner respectively to the standalone device 112. A return patient may login using the details provided earlier and authentication provided by fingerprint/retina scans. In various embodiments, the standalone device 112 may include various sensors including but not limited to sensors for weight, blood pressure, body temperature, ECG, ultrasound, pulse, blood sugar, blood test, urine test, x-ray, eye check, and the like. The stand alone device 112 may also include other paraphernalia required to collect certain physical details of a patient including but not limited to a camera, fingerprint scanner, retina scanner, printer, microphone, speaker, a touch screen and the like, and a medical storage compartment. Once logged in, patient can use all the required sensors and urine, blood samples, etc. . . . The speaker/touch screen may provide voice and/or video instructions/information and guide patient on how to place these sensors on the patient, on how to provide blood, urine, etc . . . samples in the standalone device 112. The standalone device 112 may then process the blood, urine, etc . . . samples, collect the required vitals of the patient, and send the processed information to the server 110. The standalone device 112 may automatically suggest BMI details depending upon the information provided by the patient while registering. The standalone device 112 may include a designated software program that may be capable of determining the need for any additional tests or details, for example, if there is a need to collect X-ray, the standalone device 112 may guide the patient accordingly to take an X-Ray photograph and send the resultant X-ray to the server 110. In various embodiments, all information gathered from a patient may be stored in the server. The standalone device 112 may then communicate with medical practitioners (one or a plurality of medical practitioners) situated in various different parts of the country and world via the server. In one embodiment, one of the available doctor may pick up the call and get all the details sent and processed by standalone device 112, for example, blood report, urine report, and other vitals. The medical practitioner may also get pictures of patient and any other patient information, sent by the standalone device 112. The medical practitioner may receive the information on the standalone mobile device 118 and or the standalone computer device 120. In embodiments, where the medical practitioner's diagnosis indicates that the patient's symptoms are non severe, for example, a viral or bacterial infection or not life threatening, the medical practitioner may prescribe a medicine to the patient, the prescription for which will be sent to the standalone device 112. If the medicine is available in the standalone device 112 then the standalone device 112 may dispense the medicine after collecting a payment from the patient. In certain embodiments, the standalone device may forfeit the payment dispense the medicine, for example, for charity. In case the medicine is not available in the standalone device 112, the request may be sent to the nearby storage warehouse 122 from where it may be delivered either manually or via drone or unmanned vehicles 124. This will speed up the delivery. In certain embodiments, the patient may be enabled to communicate with a medical practitioner on a video call or phone call using the standalone device 112. The standalone device 112 may also be capable of providing receipts for the payment, either a physical receipt printed by a printer included in the standalone device 112 or an e-mail receipt sent to any one of the devices used by the patient via the server 110. In certain embodiments, a medical report may be generated with the medical details and diet chart (if needed), which may be stored in the server, and the printer may be instructed to print the prescription for the patient.

In an embodiment, where a location may not have internet connection, the standalone device 112 may perform in standalone mode. The standalone device 112 in standalone mode may not be directly in communication i.e., directly connected to any server. The standalone device 112 in standalone mode may include a database to house the patient information i.e., details and medical information of the patient. In certain embodiments, the patient information may be collected using a plug in device, for example, a USB, and the plug in sent to the medical practitioner. In standalone mode, the standalone device 112 may not be enabled to communicate with remote medical practitioners. In one embodiment, the standalone device 112 in standalone mode may include heuristic capabilities and may make an attempt to provide over the counter medicine based on the patient vitals and analysis results obtained from the various sensors. In an embodiment, if the standalone device 112 in standalone mode determines that no over the counter medicine is helpful or if the medical kiosk runs out of a required medication then the medical kiosk may recommend that the patient visit medical practitioners in the vicinity of the patient and direct the patients to the medical practitioners.

In one embodiment, the standalone device 112 may be designed in a portable form. A user may purchase the portable form to be used at home. The user/patient may be enabled to connect to a medical practitioner via the server when there is a need. In one embodiment the portable version may contain all sensors and paraphernalia discussed above other than the medical storage compartment. In one embodiment, the portable standalone device may include the standalone personal device 114 in FIG. 1 which may include all sensors. In one embodiment, the portable standalone device may include the standalone mobile device 116 with a designated computer program and an option of being connected to portable sensors.

It may be appreciated by a person with ordinary skill in the art, in light of and in accordance with the teachings of the present invention, that the standalone device 112 may be virtually any electronic device capable of housing paraphernalia required to collect certain physical details of a patient including but not limited to a camera, fingerprint scanner, retina scanner, printer, microphone, speaker, a touch screen and the like; various sensors including but not limited to sensors for weight, blood pressure, body temperature, ECG, ultrasound, pulse, blood sugar, blood test, urine test, x-ray, eye check, and the like; and a medical storage compartment. In one embodiment, the standalone device 112 may include a medical kiosk.

It may be appreciated by a person with ordinary skill in the art, in light of and in accordance with the teachings of the present invention, that the set of devices of the patient 112, 114, 116 and the set of devices of the medical practitioner 118, 120 may connect to any number of devices with virtually any wired and/or wireless means. The set of devices of the patient and the medical practitioner may connect to virtually any device by means such as, but not limited to, Bluetooth connection, ethernet cable, USB cable, WIFI, IRDA, etc. In an alternative embodiment of the present invention the set of devices may send additional patient information to a server and/or to another standalone device via a cellular connection.

It may be appreciated by a person with ordinary skill in the art, in light of and in accordance with the teachings of the present invention, the standalone devices 116, 118 may include a smartphone. In an embodiment, the smartphone in use by a patient 116, the smartphone may be any device capable of connecting to various sensors, executing the designated software program, capturing information on a health status of a patient, and transmitting the information to the server 110. In an embodiment, the smartphone in use by a medical practitioner 118, the smartphone may be any device capable of executing the designated software program, and enabling a medical practitioner to receive from the server 110 and read the information a health status of a patient, and provide necessary guidance/prescription to the patient via the server 110.

It may be appreciated by a person with ordinary skill in the art, in light of and in accordance with the teachings of the present invention, a server 110 may be virtually any computing platform such as, but not limited to, a computer cluster, a laptop, a tablet, a smartphone, a cloud server, etc. . . . In another embodiment of the present invention, server 110 may be a computer connected to smartphone 116/118 via a USB cable.

It may be appreciated by a person with ordinary skill in the art, in light of and in accordance with the teachings of the present invention, a database (not shown in FIG. 1) may be virtually any data storage device or devices. Database may be, but not limited to, a plurality of data servers, a memory card. In another embodiment of the present invention, database may be a memory card connected to server 110.

It may be appreciated by a person with ordinary skill in the art, in light of and in accordance with the teachings of the present invention, that database may contain virtually any data to improve the functionality of the medical kiosk services. Database may include, without limitation, symptom tables, resolution libraries, medicine lists, test lists, medical device lists, image databanks, and sound databanks.

It may be appreciated by a person with ordinary skill in the art, in light of and in accordance with the teachings of the present invention, system of medical kiosk services 100 may partially or completely contained in a local computing platform and/or network. In an alternative embodiment of the present invention, system of medical kiosk services 100 may be located on a local computer network.

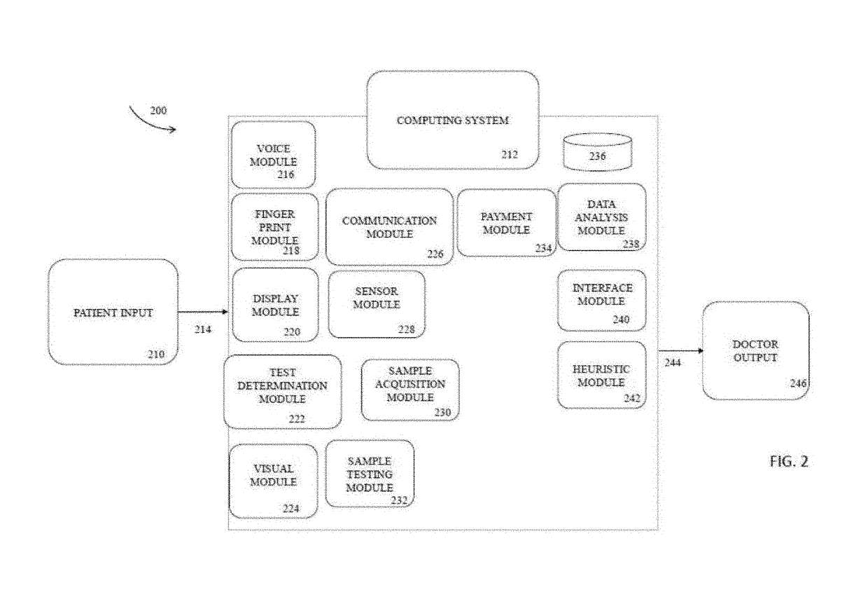

Referring to FIG. 2 is illustrated an architecture of an exemplary medical kiosk service system, in accordance with an embodiment of the present invention. A medical kiosk service system architecture 200 may comprise a computing system 212 included in a standalone device 112, i.e., a medical kiosk. The computing system 212 may include a voice module 216, a finger print module 218, a display module 220, a test determination module, a visual module 224, a communication module 226, a sensor module 228, a sample acquisition module 230, a sample testing module 232, a payment module 234, a database 236, a data analysis module 238, an interface module 240, and a heuristic module 242. Voice module 216 may have a means of receiving a voice input, processing the voice input, and providing a voice input such as, without limitation, a processing unit, a computer, or a server 110 to execute computer code and/or algorithms from a non-transitory computer readable medium for a patient's, medical practitioners, or standalone device's verbal input/output. Finger print module may have a means of recording, detecting, authenticating a finger print on the standalone device 112, such as, without limitation, sensors and/or a processing unit, and may be displaying a finger print recorded, detected, and/or authenticated message on a display module 220. A display module 220 may have a means of displaying an image, for example, image of guidance on how to use sensors (stationary or video), test results, x-ray image, ultrasound image, image of a patient or parts of the patient's body, and the like. A test determination module may have a means of determining a test for the patient to undergo such as, without limitation, a processing unit, a computer, or a server 110 to execute computer code and/or algorithms from a non-transitory computer readable medium for test determination. A visual module 224 may have a means of receiving a visual input, processing the visual input, and providing a visual output such as, without limitation, a processing unit, a computer, or a server 110 to execute computer code and/or algorithms from a non-transitory computer readable medium for a patient's, medical practitioners, or standalone device's visual/image input/output. A communication module 226 may have a means of receiving a communication, processing the communication, and sending a communication, such as, without limitation, a processing unit, a computer, or a server 110 to execute computer code and/or algorithms from a non-transitory computer readable medium for a patient's, medical practitioners, or standalone device's communication input/output on test results, images, payment, additional tests, and the like. A sensor module 228 may have a means of receiving patient input and directing and guiding the patient to the appropriate sensors, such as, without limitation, a processing unit, a computer, or a server 110 to execute computer code and/or algorithms from a non-transitory computer readable medium to collect patient vitals. A sample acquisition module 230, may have a means of receiving patient input and directing and guiding the patient to provide the appropriate sample in the appropriate portion of the standalone device 112, such as, without limitation, a processing unit, a computer, or a server 110 to execute computer code and/or algorithms from a non-transitory computer readable medium. A sample testing module 232, may have a means of receiving patient samples and directing and guiding the standalone device 112 to perform the appropriate tests, such as, without limitation, a processing unit, a computer, or a server 110 to execute computer code and/or algorithms from a non-transitory computer readable medium. A payment module 234, may have a means of calculating and receiving the appropriate payment from a patient for the tests/analysis/medications provided by the standalone device, such as, without limitation, a processing unit, a computer, or a server 110 to execute computer code and/or algorithms from a non-transitory computer readable medium, and providing a receipt to the patient/user for the payment made. A database 236 may have a means of storing the data on the patient, test results of the patient provided by the various sensors, such as, without limitation, a processing unit, a computer, or a server 110 to execute computer code and/or algorithms from a non-transitory computer readable. In one embodiment, the database 236 may be as described herein above with reference to FIG. 1. A data analysis module 238, may have a means of analyzing the data on the test results of the patient provided by the various sensors, such as, without limitation, a processing unit, a computer, or a server 110 to execute computer code and/or algorithms from a non-transitory computer readable medium, and providing an output to the medical practitioner to make a decision on the medication. An interface module 240 may have a means of interfacing with the various modules, for example, the sensor module 228, the sample acquisition module 230, the sample testing module 232, and the data analysis module 238 and providing a combined test result, such as, without limitation, a processing unit, a computer, or a server 110 to execute computer code and/or algorithms from a non-transitory computer readable medium, and providing a combined output to the medical practitioner to make a decision on the medication for the patient 212 and provide interface support to assist the medical kiosk service system in performing its various functions. A heuristic module 242 may have a means may have a means of self-learning, such as, without limitation, a processing unit, a computer, or a server 110 to execute computer code and/or algorithms from a non-transitory computer readable medium, to assist the assimilation of patient input, sensor input, test inputs, test analysis, medical practitioner input and output, correlation between symptoms, diagnosis, and medication and may provide a combined output to the medical practitioner/patient/standalone device 112 to make a decision on the medication for the patient 212. In certain embodiments, when the standalone device 112 may be in a standalone mode where there is no internet connection, the data analysis module 238 along with the heuristic module 242 may provide a prescription/list of medical practitioners in the vicinity to the patient.

It may be appreciated by a person with ordinary skill in the art, in light of and in accordance with the teachings of the present invention, that one or more modules may be embodied in a single device. In an alternative embodiment of the present invention, all modules except the data analysis module may be embodied in a standalone device 112 which would be capable of taking a sample, providing test results of the sample, and providing the results to a data analysis center for further analysis that may enable the medical practitioner to suggest and dispense appropriate medication to a patient.

It may be appreciated by a person with ordinary skill in the art, in light of and in accordance with the teachings of the present invention, that virtually any algorithm and/or computer code may be used to recognize a visual on the voice module 216. Voice recognition algorithms and/or methods may include, without limitation, Bayesian networks, fuzzy logic, neural networks, template matching, Hidden Markov models, machine learning, data mining, feature extraction and data analysis/statistics, optical character recognition, etc. In an alternative embodiment of the present invention, a binary search tree may be implemented to extract data from a voice.

It may be appreciated by a person with ordinary skill in the art, in light of and in accordance with the teachings of the present invention, that virtually any algorithm and/or computer code may be used to recognize a visual on the visual module 224. Visual recognition algorithms and/or methods may include, without limitation, Bayesian networks, fuzzy logic, neural networks, template matching, Hidden Markov models, machine learning, data mining, feature extraction and data analysis/statistics, optical character recognition, etc. In an alternative embodiment of the present invention, a binary search tree may be implemented to extra data from a visual.

It may be appreciated by a person with ordinary skill in the art, in light of and in accordance with the teachings of the present invention, that there may be a plurality of the same modules in the medical kiosk service system architecture 200. A plurality of modules such as, without limitation, voice module 216, a finger print module 218, a display module 220, a test determination module, a visual module 224, a communication module 226, a sensor module 228, a sample acquisition module 230, a sample testing module 232, a payment module 234, a database 236, a data analysis module 238, an interface module 240, and a heuristic module 242, may be present in the medical kiosk service system architecture 200. The plurality of similar modules may work in parallel or independently to improve the throughput and/or speed of the medical kiosk service system architecture 200. In an alternative embodiment of the present invention, a plurality of communication, sensor etc . . . modules may be connected to a medical kiosk service system via wired and wireless connections to access resources from different wired and wireless networks. In still another alternative embodiment of the present invention, a plurality of similar modules may form a secondary medical kiosk service system capable of seamlessly substituting a messing and/or failing module.

It may be appreciated by a person with ordinary skill in the art, in light of and in accordance with the teachings of the present invention, that one or more modules may transmit module failure to a tech support server that is on an accessible network or over the internet. In an alternative embodiment of the present invention, sensor information and patient information may be sent to a server to alleviate processing load on a medical kiosk service system.

It may be appreciated by a person with ordinary skill in the art, in light of and in accordance with the teachings of the present invention, that any module in medical kiosk service system architecture 200 may perform data manipulation. Data manipulation such as, but not limited to, compression, encryption, formatting. In an alternative embodiment of the present invention, any module sending data may first compress the data prior to data transmission.

It may be appreciated by a person with ordinary skill in the art, in light of and in accordance with the teachings of the present invention, that the standalone devices 114, 116, 118, 120 may include few or all the modules as described herein, such that the standalone devices perform similar functions and provide similar output as the standalone device 112.

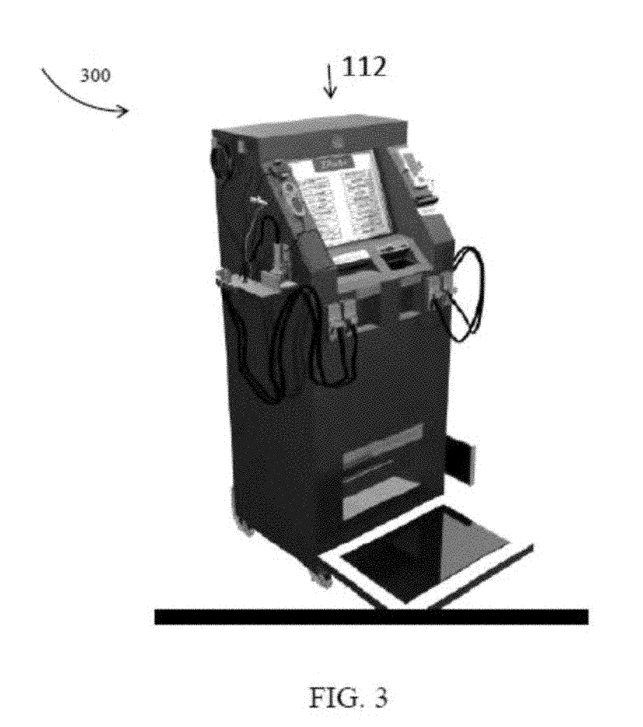

Referring to FIG. 3 is illustrated, an exemplary medical kiosk service system 300, in accordance with an embodiment of the present invention. In one embodiment, the exemplary medical kiosk 300 is the stand-alone device 112 shown in FIG. 1.

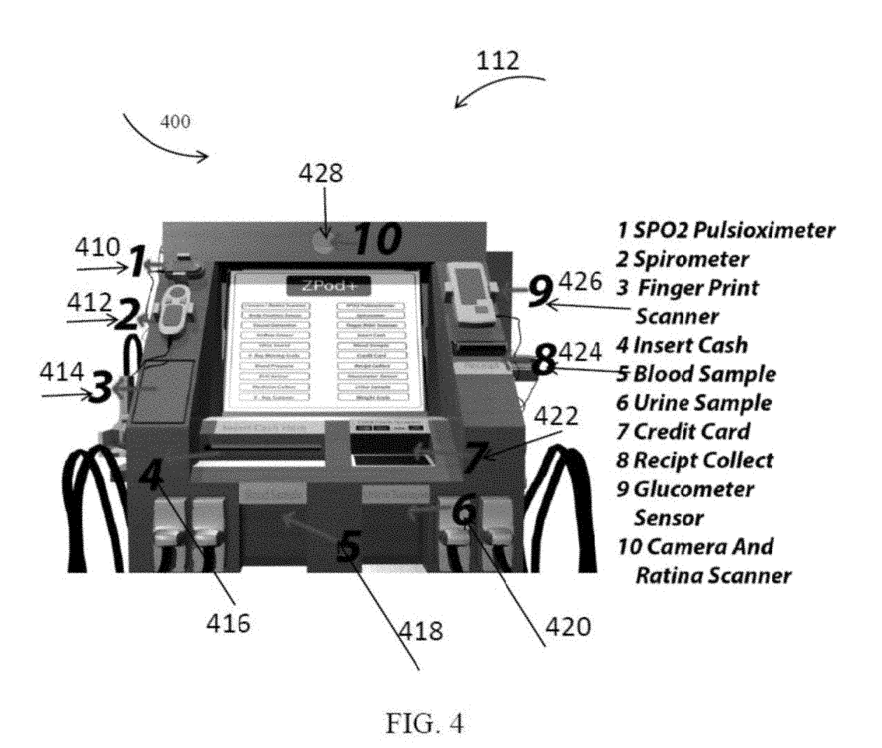

Referring to FIG. 4 is illustrated a portion of an exemplary medical kiosk service system 400, in accordance with an embodiment of the present invention. As shown in FIG. 4, the stand-alone device 112, in one embodiment may include, but is not limited to, an SPO₂ pulsioximeter 410, a spirometer 412, a finger print scanner 416, a cash receiving port 418, a blood sample receiving port 418, a urine sample receiving port 420, a payment (for example, credit card, debit card, health card, cash, and the like) accepting port 422, a receipt providing port 424, a glucometer sensor 426, voice scanner, and a camera and retina scanner on the front portion of the standalone device 112. It may be appreciated by a person with ordinary skill in the art, in light of and in accordance with the teachings of the present invention, that any scanner or port may be placed in any portion of the standalone device 112 as long as it is conveniently accessible to a patient or user for performing the necessary actions.

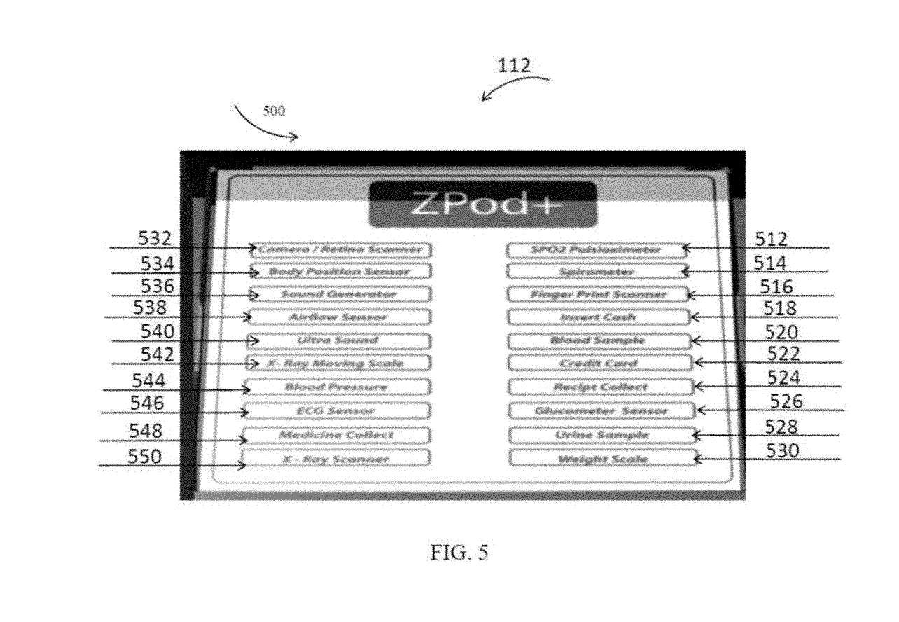

FIG. 5 illustrates a portion of an exemplary medical kiosk service system 500, in accordance with an embodiment of the present invention. FIG. 5 illustrates the display 510 of the standalone device 112. In one embodiment, the display 510 may provide a patient or user with links that will guide the patient to provide the requisite samples in the appropriate ports, and use the appropriate sensors in an appropriate manner to provide patient vitals or use the appropriate links to pay, collect receipt, collect medicine, etc. . . . In one embodiment, the display 510 may include a touch screen. In another embodiment, the patient may be enabled with a key pad/keyboard/mouse to navigate the various links provided in the display. In one embodiment, as shown in FIG. 5, the links may include but are not limited to SP02 pulsioximeter 512, spirometer 514, finger print scanner 517, insert cash 518, blood sample 520, credit card 522, receipt collect 524, glucometer sensor 526, unite sample 528, weight scale 530, camera/retina scanner 532, body position sensor 534, sound generator 536, airflow sensor 538, ultra sound 540, X-ray moving scale 542, blood pressure 544, ECG sensor 546, medicine collect 548, X-ray scanner 550, etc . . . It may be appreciated by a person with ordinary skill in the art, in light of and in accordance with the teachings of the present invention, that the links may be all provided in a single page display or in a multiple page display. In one embodiment, the links may be grouped into sample collections, sensors, payment/receipts, medicine collection, etc . . . In one embodiment, the links may be provided in any language depending on the location where the kiosk is placed. In one embodiment, the medical kiosk may also provide brail language, voice input, etc . . . for the convenience of differently abled users/patients.

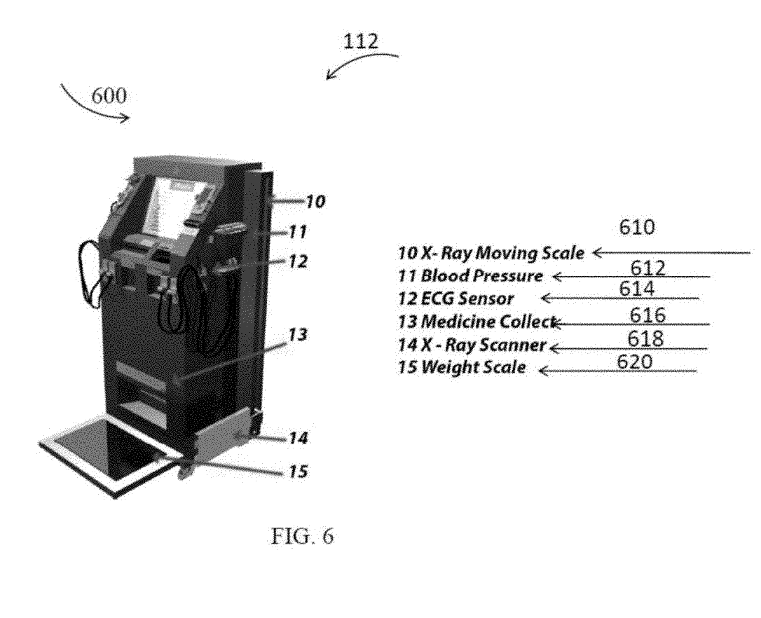

Referring to FIG. 6 is illustrated a portion of an exemplary medical kiosk service system 600, in accordance with an embodiment of the present invention. In the view shown in FIG. 6, the standalone device 112 may include an X-ray moving scale 610, a blood pressure sensor 612, a ECG sensor 614, a medicine dispensing/collection port 616, an X-ray scanner 618, and a weight scale 620. It may be appreciated by a person with ordinary skill in the art, in light of and in accordance with the teachings of the present invention, that any scanner or port may be placed in any portion of the standalone device 112 as long as it is conveniently accessible to a patient or user for performing the necessary actions.

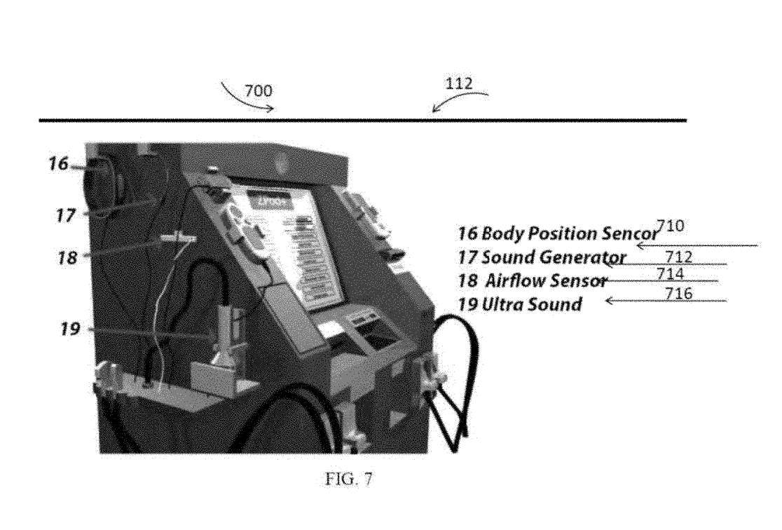

FIG. 7 illustrates a portion of an exemplary medical kiosk service system 700, in accordance with an embodiment of the present invention. In the view shown in FIG. 7, the standalone device 112 may include a body position sensor 710, a sound generator 712, an airflow sensor 714, and an ultrasound device 716.

In one embodiment, the standalone device 112 may include a remotely controlled ultrasound device. In one embodiment, the ultrasound device may include a belt, which may be hollow, and may contain a remotely controlled movable block with attached ultrasound probe. The probe may move up and down based on the commands given by a remote system which may be controlled by a medical practitioner who is remotely located. The medical practitioner may also control the movable block, enabling the medical practitioner to guide the probe to the right place and move up and down to put proper pressure to get the best possible ultrasound images. It may be appreciated by a person with ordinary skill in the art, in light of and in accordance with the teachings of the present invention, that the belt may be made available in various sizes, shapes, and materials suitable for different parts of body.

In one embodiment, the standalone device 112 may include a portable X-ray with movable scanner. The X-ray scanner may be attached to any physical portable unit which can move up and down or side ways to take the best possible picture of a body part. In multiple embodiments, the patient may either be in a standing position or in a sleeping position. The X-rays may then be sent to the server for analysis.

As used herein, the term medical practitioner may include a doctor, nurse, or a technician trained and authorized by a certifying authority, and the like.

In one embodiment, is provided a first version of the medical kiosk that may include sensors for all body vitals and remote doctor connectivity. In this version, the kiosk may include all the sensors attached to the system and an interactive screen to communicate with the remote doctors. This version may be enabled to collect the body vitals, send to the doctor, and the doctor may prescribe the medicines. It may include a medicine dispenser which may work based on doctor's prescription. This dispenser may store various common medicines which doctor may prescribe. If the medicine is available in the kiosk then upon doctor's prescription medicine may be delivered. If the medicine is not available in the kiosk then the system may send a request to a central warehouse from where the medicine may be either sent by automated drone, unmanned vehicle or by service vehicle.

In another embodiment, is provided a second version of the medical kiosk which may include a kiosk with built in ultra sound and x-ray. In this version, the kiosk may have all the features included in version 1, and have additional devices including ultra sound and X-ray machines. The ultra sound prob may be controlled remotely by remote doctors or technician to get ultra sound images of various parts of the body. In one embodiment, the X-ray machine may be portable and attached to the kiosk which can move up, down and sidesways such that the patient may stand or lie down.

In yet another embodiment, is provide a third version of the medical kiosk which may include builtin ultra sound, X-ray, blood, and urine sample processors. This version may include all the features from version 2 and in addition, may include blood and urine sample processor which may allow patients to put their samples in the system and system will then process and send the results to the server/doctors for analysis.

In one embodiment, the standalone device 112 may be made of a combination of metal and plastic materials and in certain embodiments may resemble a an automated teller machine (ATM). It may include all the sensors mentioned herein, X-ray unit, ultrasound unit, blood and urine testing unit with required chemicals for testing, tools for authentication like fingerprint scanner and retina scanner, a touch screen, microphone, speakers and headphones, USB, and HDMI jack.

In one embodiment, all the sensors may be controlled via system based on the standard defined protocol or workflow by a panel of expert doctors. System may collect blood sample and urine sample and perform the required tests instructed by the system. If ultrasound or x-ray is required then system may direct the user and instruct the patient via a video or on screen instructions on how to use the sensors.

In various embodiments, all the sensors used in the system may be those readily available in the market. For example, blood pressure sensor, ECG sensor, airflow breathing sensor, body temperature sensor, SPO2 pulse oxygen, glucometer Sensor, and the like.

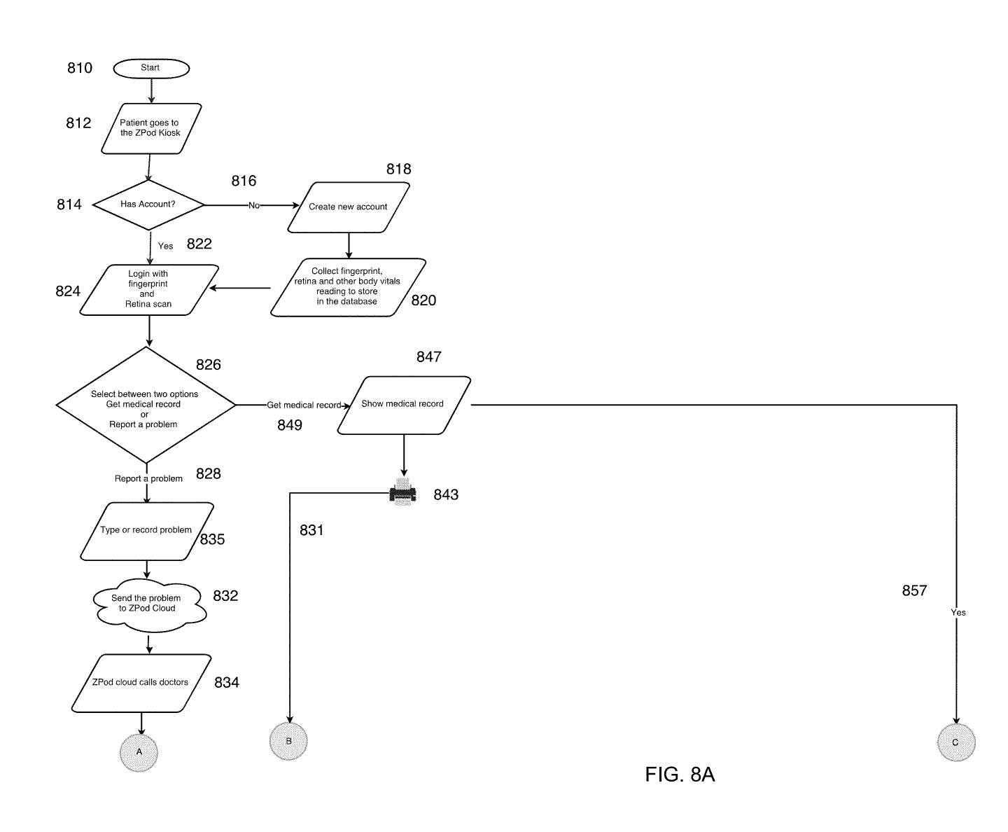

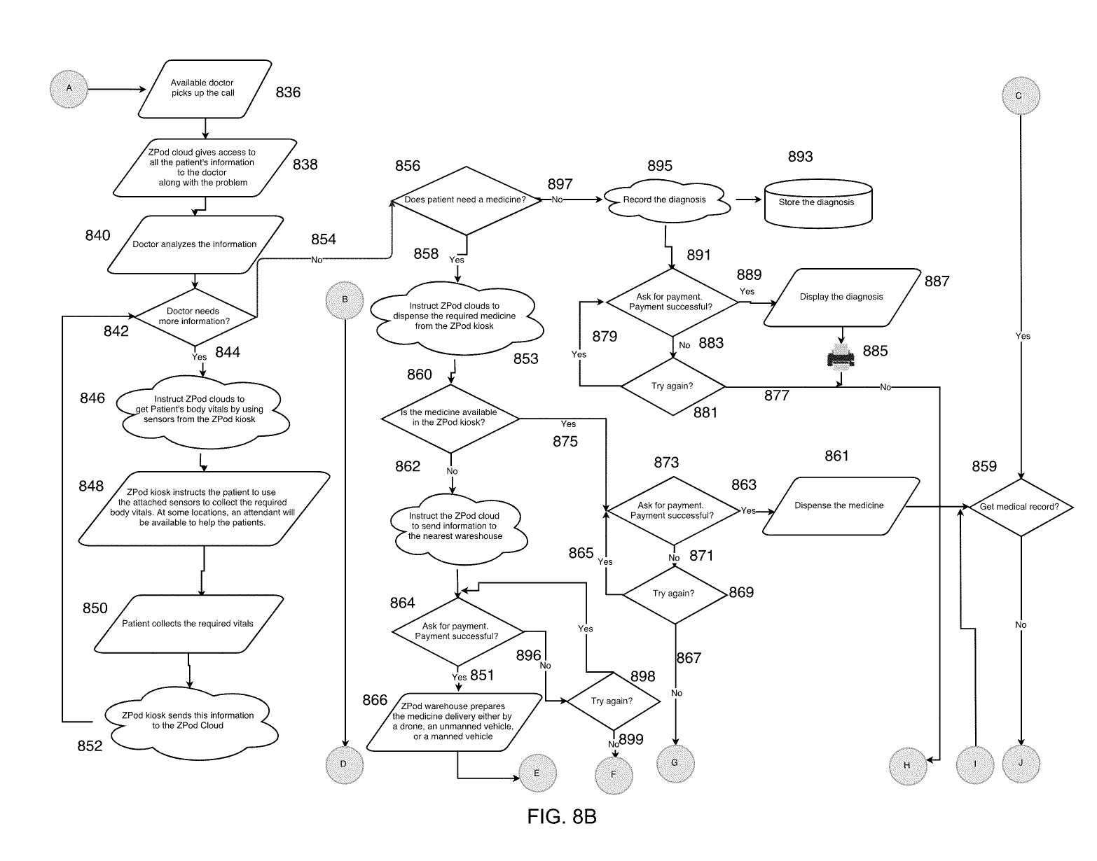

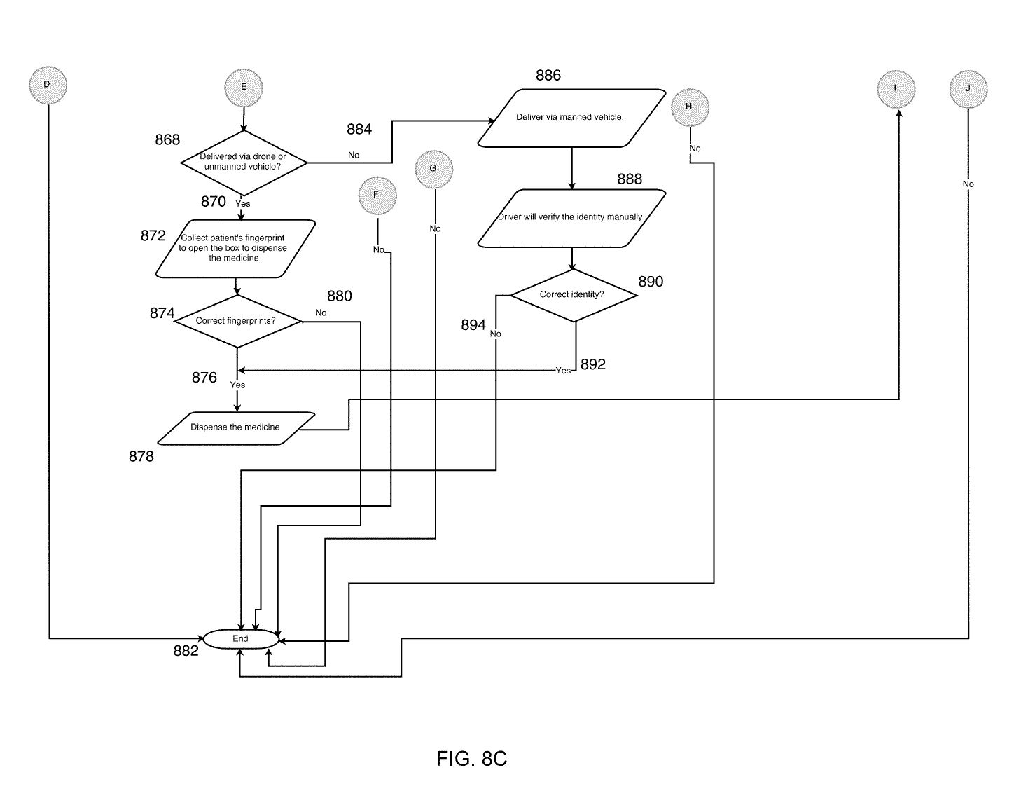

FIGS. 8A through 8C is a flow chart 800 illustrating an exemplary process for using a medical kiosk service system, in accordance with an embodiment of the present invention. In embodiments described herein, the medical kiosk service system may include a public or personal version. In the public version embodiment, a process starts with a step 810. In a step 812 a patient may go to a nearby medical kiosk service system location. In a step 814 the patient may have to evaluate if the patient has an account with the medical kiosk service system. If “NO” 816, and if it is the first time the patient is visiting the medical kiosk service system the system may guide and allow the patient to create an account in step 818. In step 820 the system may collect the patient's fingerprint scan, retinal images, and system may ask basic details about the patient and instruct the to make use of sensors for recording various patient vitals like blood pressure, blood sugar, height, weight, hair color, ethnicity, etc . . . If “YES” 822, patient already has an account in the system, the patient may use finger print and retina scanner in 824 step to login to the system. It may be appreciated by a person with ordinary skill in the art, in light of and in accordance with the teachings of the present invention, that other known secure methods of logging in may also be provided in the medical kiosk, for example, password, voice recognition, and the like.

Once the patient has logged in, in step 826 the patient may be provided with an option to either check the patient's medical record or report a current problem. If the patient chooses to report a problem 828, in a step 830 the patient may record the problem. It may be appreciated by a person with ordinary skill in the art, in light of and in accordance with the teachings of the present invention, that the patient may record the problem using various methods including but not limited to keying in the details, recording the details, scanning the details, etc . . . In a step 832 the details provided by the patient may be sent to the server 110, i.e., once the details are entered/recorded and the patient hits the ‘send’ button and medical kiosk may then send that data to the server. In one embodiment, if a patient is not well, an authorized user may include the symptoms of the patient or record the patient's problem and submit it in place of the patient. Once the information is submitted, medical kiosk service system's server 110, in step 834, may try to communicate with all the active medical practitioners who may be registered in medical kiosk service system as service providers. In various the embodiments, the medical practitioner may receive a call, text message, or email on their standalone mobile device 118 or their standalone computer device 120 from the server 110. In step 836 any medical practitioner who is available to help the patient may accept the call. In step 838 the medical practitioner who accepts the call may get all the information including recorded or typed message and all the details about the patient. In a step 840 the medical practitioner may analyze the information. In step 842 the medical practitioner may determine if medical assistance may be provided to the patient using the available information or if more information is needed to provide the medical assistance. If “YES” 844 i.e., more information is needed, in one embodiment, the medical practitioner may send instructions to the patient through the server 110, requesting for patient's vital information. In step 846, the server 110 may instruct the patient to use the attached sensors and collect the required body vitals. In certain embodiments, an attendant may be available to assist a patient to use the sensors and collect the body vitals. In one embodiment, the medical practitioner may make a video call to talk to the patient. The patient may receive the video call on the standalone device 112 of the medical kiosk service system. In this embodiment, the medical practitioner may have a face to face verbal communication with the patient, which may be recorded and stored in the patient's file in the server 110 and the database. If the medical practitioner needs more readings the medical practitioner may instruct the patient to use the relevant sensor to record the reading. For example, if blood or urine sample is needed then medical practitioner may instruct the standalone device 112 of the medical kiosk service system to accept the samples. The patient may then insert the tubes filled with either blood or urine into the blood sample section or urine sample section. The standalone device 112 may then process the sample. In step 850 the patient may collect the required vitals. In step 852 the standalone device 112 may send the vitals collected in step 850 to the server 110. The server 110 may then send the readings to the medical practitioner and store it in the patient's database. In certain embodiments, if the medical practitioner is not in a position to determine or diagnose the health status of the patient, the patient may be referred to a nearby hospital for further evaluation. It may be appreciated by a person with ordinary skill in the art, in light of and in accordance with the teachings of the present invention, that the medical kiosk system may or may not include or be affiliated to a predefined list of hospitals. In an event where the medical kiosk may be affiliated to a predefined list of hospitals, the server may transfer the patient data to the hospital and/or give patient an option to print the diagnosis to take with him/her to the hospital. In an event where the medical kiosk system may not be affiliated with a predefined list of hospitals, the patient may be enabled to take a print of the diagnosis and take the diagnosis to a hospital. After the treatment, patient may send the report to the server for updating patient information. If “NO” 854, i.e., additional information is not required or if the additional information sent in step 852 is sufficient for the medical practitioner to make a diagnosis and provide medical assistance to the patient, the medical practitioner in step 856 determine if a medicine needs to be prescribed to the patient. If “YES” 858 i.e., medicine needs to be prescribed, the medical practitioner may send an instruction to the server 110 to dispense the required medication from the standalone device 112 in step 853. In step 860 the standalone device 112 may determine if the prescribed medication is available in the medication dispenser. If “NO” 862, the medication is not available then the standalone device 112 may instruct the server 110 to send the information to a nearest warehouse. The standalone device 112 may then request the patient to make a payment in step 864. If “YES” 851 i.e., payment is successful the warehouse of the medical kiosk service system may instruct the warehouse to deliver the medication to the patient using a drone, manned or unmanned vehicle in step 866. In various embodiments, the patient may be enabled to make a payment using but not limited to credit card, debit card, cash, or a preloaded healthcare card. The standalone device 112 may then determine on how to deliver in step 868, i.e., using a drone, manned or unmanned vehicle. If “YES” 870, i.e., a drone or unmanned vehicle is being used then in step 872 the drone or unmanned vehicle may collect the patient's fingerprint in step 874. If the finger print is authenticated, “YES” 876 in step 878 the drone or unmanned vehicle may dispense the medicine. If the finger print is not authenticated, “NO” 880 the drone or unmanned vehicle may not dispense the medicine and the process comes to an end in step 882. In certain embodiments, a back up authentication may be provided, for example retinal scan, voice scan, password, and the like. In some embodiments, the medical dispensation at the medical kiosk may be protected with a lock. A medical practitioner may be enabled to release the key from the medical kiosk on a patient's request and the patient may be required to place the key back in the medical kiosk. In certain embodiments, the medical kiosk may have a temperature controlled box for housing the medicines, for example, storing vaccinations that may need low temperatures, etc . . . If “NO” 884, i.e., a manned vehicle is being used in step 886 a person in the manned vehicle in step 888 may verify the patient's identity. If the identity is authenticated, “YES” 892 in step 878 the manned vehicle may dispense the medicine. If the identity is not authenticated, “NO” 894 the manned vehicle may not dispense the medicine and the process comes to an end in step 882. In step 864 if the payment is not successful “NO” 896 the standalone device 112 will provide an opportunity to the patient to try again in step 898. If the payment is again unsuccessful “NO” 899 the process comes to an end in step 882. If in step 856 the medical practitioner determines that the patient may not require medication, i.e., “NO” 897 the medical practitioner may record the diagnosis in step 895 and store the diagnosis in the server or database in step 893. The standalone device 112 may then request the patient to make a payment in step 891. If “YES” 889 i.e., payment is successful the standalone device 112 may display the diagnosis on its display module and allow the patient to take a printout in step 885, further to which the process comes to an end in step 882. In step 891 if the payment is not successful “NO” 883 the standalone device 112 will provide an opportunity to the patient to try again in step 881. If the payment is successful “YES” 879 the standalone device 112 may display the diagnosis on its display module and allow the patient to take a printout in step 885, further to which the process comes to an end in step 882. If the payment is again unsuccessful “NO” 877 the process comes to an end in step 882. If in step 860, the medication is available in the standalone device 112, “YES” 875, the standalone device 112 may then request the patient to make a payment in step 873. If “YES” 873 i.e., payment is successful the standalone device 112 may dispense the medication in step 861 and the process may come to an end in step 882. In step 859, if the patient needs a medical record “YES” 857 the standalone device 112 may be requested to show the medical record in step 847 and the record printed 843 and the process may then end in step 882. In step 859, if the patient needs a medical record “NO” 855 the process may come to an end in step 882. Similarly, in step 826 if the patient decides to get a medical report in step 849 the standalone device 112 may be requested to show the medical record in step 847 and the record printed 843 and the process may then end in step 882.

It may be appreciated by a person with ordinary skill in the art, in light of and in accordance with the teachings of the present invention, that in certain embodiments, the kiosk may include educational content as the system may be used to provide for patients who may need to better understand their medical issues, find the appropriate hospitals/doctors and the like.

In certain embodiments, the personal version of the standalone device i.e., the standalone personal device 114 may include all other sensors present in the standalone device 112 other than the X-ray, blood and urine sample processor. In one embodiment, standalone personal device 114 may be connected to additional portable sensors to collect all the body vitals, blood samples, urine samples, x-ray and ultra sound. The standalone personal device 114 may also have an interactive display screen which may allow patients and remote medical practitioners to communicate with each other. This display screen may provide various other functions like checking patients data, checking previous prescriptions, etc . . .

In one embodiment, in the standalone mobile device 116 the patient may be enabled to talk to a doctor, check medical record, book appointments with specific doctors, report general problems, check frequently asked questions (FAQs) or educate themselves on various medical conditions. In one embodiment, the standalone mobile device 116 may not include any sensors. In one embodiment, the personal devices may be enabled to connect to all the sensors via communication mode like Bluetooth, wireless, etc. . . . in a plug and use manner.

In certain other embodiments, the medical kiosk system may provide reminders to the patients. In one embodiment, the server may send the reminders to the personal device of the patient. In another embodiment, the patient may access the server using the medical kiosk. If there are any reminders, then patient may be notified as soon as he/she logs in to the system and if payment is required then the medical kiosk may direct the patient to the payment page. After that it the medical system may follow the usual payment and dispensing flow as described herein above.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

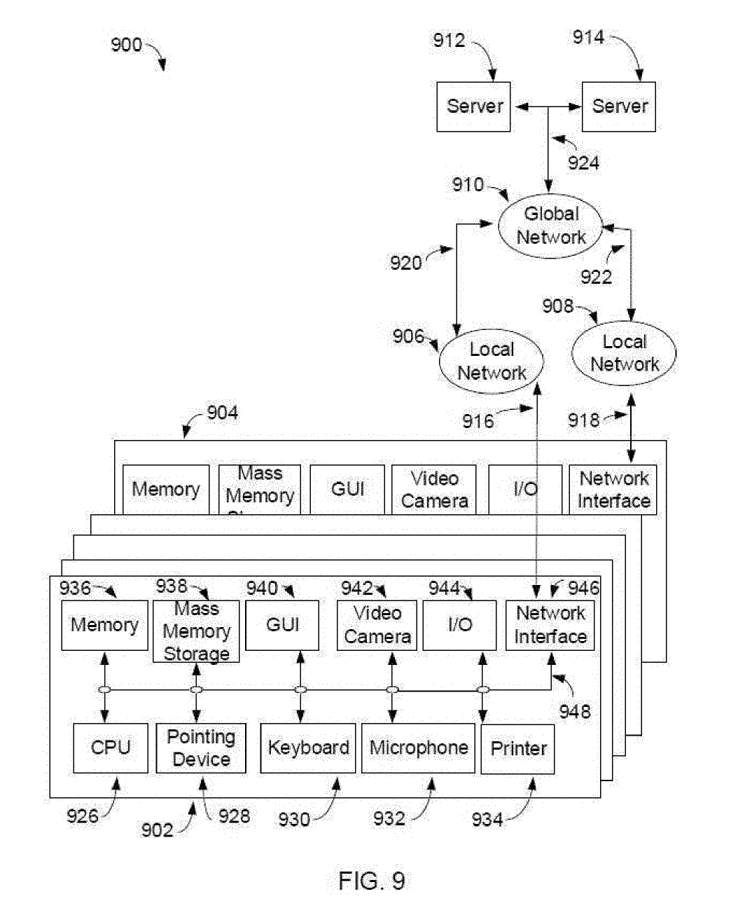

Referring to FIG. 9 is provided a block diagram depicting an exemplary client/server system which may be used by an exemplary web-enabled/networked embodiment of the present invention. A communication system 900 includes a multiplicity of clients with a sampling of clients denoted as a client 902 and a client 904, a multiplicity of local networks with a sampling of networks denoted as a local network 906 and a local network 908, a global network 910 and a multiplicity of servers with a sampling of servers denoted as a server 912 and a server 914.

Client 902 may communicate bi-directionally with local network 906 via a communication channel 916. Client 904 may communicate bi-directionally with local network 908 via a communication channel 918. Local network 906 may communicate bi-directionally with global network 910 via a communication channel 920. Local network 908 may communicate bi-directionally with global network 910 via a communication channel 922. Global network 910 may communicate bi-directionally with server 912 and server 914 via a communication channel 924. Server 912 and server 914 may communicate bi-directionally with each other via communication channel 924. Furthermore, clients 902, 904, local networks 906, 908, global network 910 and servers 912, 914 may each communicate bi-directionally with each other.

In one embodiment, global network 910 may operate as the Internet. It will be understood by those skilled in the art that communication system 900 may take many different forms. Non-limiting examples of forms for communication system 900 include local area networks (LANs), wide area networks (WANs), wired telephone networks, wireless networks, or any other network supporting data communication between respective entities.

Clients 902 and 904 may take many different forms. Non-limiting examples of clients 902 and 904 include personal computers, personal digital assistants (PDAs), cellular phones and smartphones.

Client 902 includes a CPU 926, a pointing device 928, a keyboard 930, a microphone 932, a printer 934, a memory 936, a mass memory storage 938, a GUI 940, a video camera 942, an input/output interface 944 and a network interface 946.

CPU 926, pointing device 928, keyboard 930, microphone 932, printer 934, memory 936, mass memory storage 938, GUI 940, video camera 942, input/output interface 944 and network interface 946 may communicate in a unidirectional manner or a bi-directional manner with each other via a communication channel 948. Communication channel 948 may be configured as a single communication channel or a multiplicity of communication channels.

CPU 926 may be comprised of a single processor or multiple processors. CPU 926 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 936 is used typically to transfer data and instructions to CPU 926 in a bi-directional manner. Memory 936, as discussed previously, may include any suitable computer-readable media, intended for data storage, such as those described above excluding any wired or wireless transmissions unless specifically noted. Mass memory storage 938 may also be coupled bi-directionally to CPU 926 and provides additional data storage capacity and may include any of the computer-readable media described above. Mass memory storage 938 may be used to store programs, data and the like and is typically a secondary storage medium such as a hard disk. It will be appreciated that the information retained within mass memory storage 938, may, in appropriate cases, be incorporated in standard fashion as part of memory 936 as virtual memory.

CPU 926 may be coupled to GUI 940. GUI 940 enables a user to view the operation of computer operating system and software. CPU 926 may be coupled to pointing device 928. Non-limiting examples of pointing device 928 include computer mouse, trackball and touchpad. Pointing device 928 enables a user with the capability to maneuver a computer cursor about the viewing area of GUI 940 and select areas or features in the viewing area of GUI 940. CPU 926 may be coupled to keyboard 930. Keyboard 930 enables a user with the capability to input alphanumeric textual information to CPU 926. CPU 926 may be coupled to microphone 932. Microphone 932 enables audio produced by a user to be recorded, processed and communicated by CPU 926. CPU 926 may be connected to printer 934. Printer 934 enables a user with the capability to print information to a sheet of paper. CPU 926 may be connected to video camera 942. Video camera 942 enables video produced or captured by user to be recorded, processed and communicated by CPU 926.

CPU 926 may also be coupled to input/output interface 944 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 926 optionally may be coupled to network interface 946 which enables communication with an external device such as a database or a computer or telecommunications or internet network using an external connection shown generally as communication channel 916, which may be implemented as a hardwired or wireless communications link using suitable conventional technologies. With such a connection, CPU 926 might receive information from the network, or might output information to a network in the course of performing the method steps described in the teachings of the present invention.

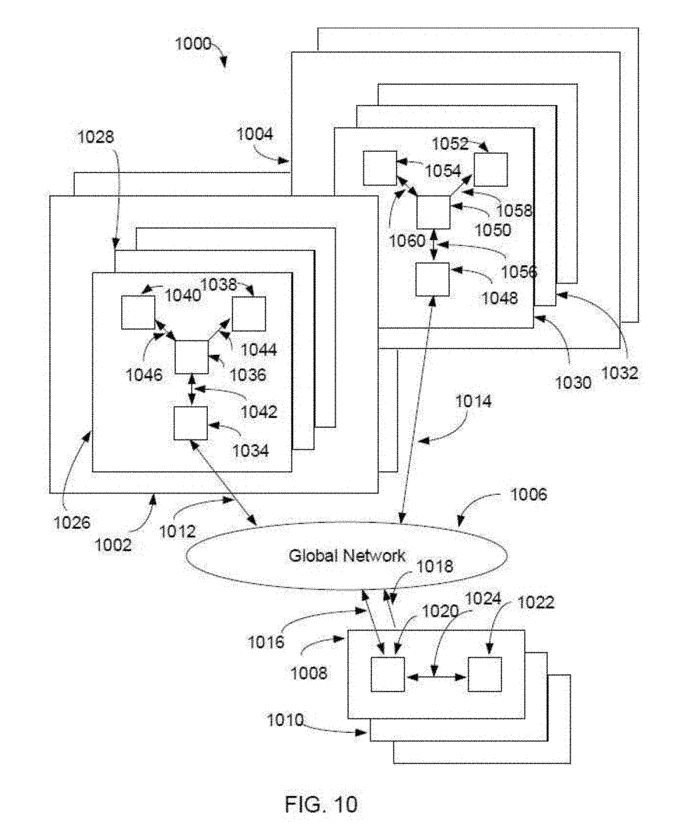

FIG. 10 illustrates a block diagram depicting a conventional client/server communication system. FIG. 10 illustrates a block diagram depicting a conventional client/server communication system, which may be used by an exemplary web-enabled/networked embodiment of the present invention.

A communication system 1000 includes a multiplicity of networked regions with a sampling of regions denoted as a network region 1002 and a network region 1004, a global network 1006 and a multiplicity of servers with a sampling of servers denoted as a server device 1008 and a server device 1010.

Network region 1002 and network region 1004 may operate to represent a network contained within a geographical area or region. Non-limiting examples of representations for the geographical areas for the networked regions may include postal zip codes, telephone area codes, states, counties, cities and countries. Elements within network region 1002 and 1004 may operate to communicate with external elements within other networked regions or within elements contained within the same network region.

In some implementations, global network 1006 may operate as the Internet. It will be understood by those skilled in the art that communication system 1000 may take many different forms. Non-limiting examples of forms for communication system 1000 include local area networks (LANs), wide area networks (WANs), wired telephone networks, cellular telephone networks or any other network supporting data communication between respective entities via hardwired or wireless communication networks. Global network 1006 may operate to transfer information between the various networked elements.

Server device 1008 and server device 1010 may operate to execute software instructions, store information, support database operations and communicate with other networked elements. Non-limiting examples of software and scripting languages which may be executed on server device 1008 and server device 1010 include C, C++, C#, PHP and Java.

Network region 1002 may operate to communicate bi-directionally with global network 1006 via a communication channel 1012. Network region 1004 may operate to communicate bi-directionally with global network 1006 via a communication channel 1014. Server device 1008 may operate to communicate bi-directionally with global network 1006 via a communication channel 1016. Server device 1010 may operate to communicate bi-directionally with global network 1006 via a communication channel 1018. Network region 1002 and 1004, global network 1006 and server devices 1008 and 1010 may operate to communicate with each other and with every other networked device located within communication system 1000.

Server device 1008 includes a networking device 1020 and a server 1022. Networking device 1020 may operate to communicate bi-directionally with global network 1006 via communication channel 1016 and with server 1022 via a communication channel 1024. Server 1022 may operate to execute software instructions and store information.

Network region 1002 includes a multiplicity of clients with a sampling denoted as a client 1026 and a client 1028. Client 1026 includes a networking device 1034, a processor 1036, a GUI 1038 and an interface device 1040. Non-limiting examples of devices for GUI 1038 include monitors, televisions, cellular telephones, smartphones and PDAs (Personal Digital Assistants). Non-limiting examples of interface device 1040 include pointing device, mouse, trackball, scanner and printer. Networking device 1034 may communicate bi-directionally with global network 1006 via communication channel 1012 and with processor 1036 via a communication channel 1042. GUI 1038 may receive information from processor 1036 via a communication channel 1044 for presentation to a user for viewing. Interface device 1040 may operate to send control information to processor 1036 and to receive information from processor 1036 via a communication channel 1046. Network region 1004 includes a multiplicity of clients with a sampling denoted as a client 1030 and a client 1032. Client 1030 includes a networking device 1048, a processor 1050, a GUI 1052 and an interface device 1054. Non-limiting examples of devices for GUI 1038 include monitors, televisions, cellular telephones, smartphones and PDAs (Personal Digital Assistants). Non-limiting examples of interface device 1040 include pointing devices, mousse, trackballs, scanners and printers. Networking device 1048 may communicate bi-directionally with global network 1006 via communication channel 1014 and with processor 1050 via a communication channel 1056. GUI 1052 may receive information from processor 1050 via a communication channel 1058 for presentation to a user for viewing. Interface device 1054 may operate to send control information to processor 1050 and to receive information from processor 1050 via a communication channel 1060.

For example, consider the case where a user interfacing with client 1026 may want to execute a networked application. A user may enter the IP (Internet Protocol) address for the networked application using interface device 1040. The IP address information may be communicated to processor 1036 via communication channel 1046. Processor 1036 may then communicate the IP address information to networking device 1034 via communication channel 1042. Networking device 1034 may then communicate the IP address information to global network 1006 via communication channel 1012. Global network 1006 may then communicate the IP address information to networking device 1020 of server device 1008 via communication channel 1016. Networking device 1020 may then communicate the IP address information to server 1022 via communication channel 1024. Server 1022 may receive the IP address information and after processing the IP address information may communicate return information to networking device 1020 via communication channel 1024. Networking device 1020 may communicate the return information to global network 1006 via communication channel 1016. Global network 1006 may communicate the return information to networking device 1034 via communication channel 1012. Networking device 1034 may communicate the return information to processor 1036 via communication channel 1042. Processor 10106 may communicate the return information to GUI 10108 via communication channel 1044. User may then view the return information on GUI 1038.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC § 112 (6) it is intended that the corresponding means for and/or steps for carrying out the claimed function are the ones that are locally implemented within the jurisdiction of the USA, while the remaining aspect(s) performed or located remotely outside the USA are not intended to be construed under 35 USC § 112 (6).]

It is noted that according to USA law, all claims must be set forth as a coherent, cooperating set of limitations that work in functional combination to achieve a useful result as a whole. Accordingly, for any claim having functional limitations interpreted under 35 USC § 112 (6) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC § 112 (6) is/(are) dependent on one or more remote servers located outside the USA, it is intended that each such recited function under 35 USC § 112 (6) is to be interpreted as the function of the local system receiving the remotely generated information required by a locally implemented claim limitation, wherein the structures and or steps which enable, and breath life into the expression of such functions claimed under 35 USC § 112 (6) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present application, and “35 USC § 112 (6)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112 (6) when such corresponding structures are not explicitly disclosed in the foregoing patent specification. Therefore, for any invention element(s)/structure(s) corresponding to functional claim limitation(s), in the below claims interpreted under 35 USC § 112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing medical kiosk service system according to the present invention will be apparent to those skilled in the art. Various aspects of the invention have been described above by way of illustration, and the specific embodiments disclosed are not intended to limit the invention to the particular forms disclosed. The particular implementation of the medical kiosk service system may vary depending upon the particular context or application. By way of example, and not limitation, the medical kiosk service system described in the foregoing were principally directed to providing automated medical services particularly in remote areas; however, similar techniques may instead be applied to cosmetics, veterinary medicines, Psychiatry, Nursing homes, Rehabilitation centers, Mental health institutions, Dermatology, Sports Medicine, Vision Therapy, Orthopedics, Physiotherapy, Pediatrics, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

What is claimed is:

1. A method comprising the steps of:
providing a first standalone device, wherein the first standalone device is configured for interacting with a patient and capturing details and medical information of the patient, wherein the first standalone device comprises a plurality of sensors and devices for capturing details and medical information of the patient;
providing a server in communication with the first standalone device, wherein the first standalone device is configured to communicate the medical information of the patient to the server, wherein the server stores the medical information of the patient;
providing a second standalone device, configured for interacting with a medical practitioner;
the server in communication with the second standalone device, wherein the server is configured to communicate the medical information of the patient to the second standalone device;
wherein a diagnosis is communicated by the second standalone device to the first standalone device via the server;
wherein the first standalone device is configured to provide the patient with a prescription or a medication.

2. The method of claim 1, wherein the first standalone device is a medical kiosk.

3. The method of claim 2, wherein the medical kiosk houses the plurality of sensors and devices for capturing details and medical information of the patient

4. The method of claim 1, wherein the first standalone device is a standalone personal device comprising the plurality of sensors and devices for capturing details and medical information of the patient.

5. The method of claim 1, wherein the first standalone device is a standalone mobile device configured for connect to a plurality of portable sensors and devices for capturing details and medical information of the patient.

6. The method of claim 1, wherein the plurality of sensors and devises comprise: an SPO₂ pulsioximeter, a spirometer, a finger print scanner, a cash receiving port, a blood sample receiving port, a urine sample receiving port, a payment accepting port, a receipt providing port, a glucometer sensor, a camera and retina scanner on the front portion of the standalone device, a voice scanner, X-ray scanner, ultrasound scanner, and the like.

7. The method of claim 1, wherein the second standalone device comprises a computer device with a designated software program to enable a medical practitioner to check patient record, prescribe medicine, and the line.

8. The method of claim 1, wherein the prescribed medication is collected from a medicine dispensing port connected to a medicine storage in the first standalone device, or delivered using a manned or unmanned vehicle, or delivered using drones.

9. The method of claim 1, wherein the first standalone device provides instructions to the patient for self-using the plurality of sensors and devices.

10. The method of claim 1, wherein the first standalone device comprises a database to house the details and medical information of the patient, wherein the first standalone device is not directly connected to the server, and the information is communicated to the server using a plugin device.

11. A system comprising:
a first standalone device, configured for interacting with a patient and capturing details and medical information of the patient, wherein the first standalone device comprises a plurality of sensors and devices for capturing details and medical information of the patient'
a server in communication with the first standalone device, wherein the first standalone device is configured to communicate the medical information of the patient to the server, wherein the server stores the medical information of the patient;
a second standalone device, configured for interacting with a medical practitioner;
the server in communication with the second standalone device, wherein the server is configured to communicate the medical information of the patient to the second standalone device;
wherein a diagnosis is communicated by the second standalone device to the first standalone device via the server;
wherein the first standalone device is configured to provide the patient with a prescription or a medication.

12. The system of claim 11, wherein the first standalone device is a medical kiosk.

13. The system of claim 12, wherein the medical kiosk houses the plurality of sensors and devices for capturing details and medical information of the patient

14. The system of claim 11, wherein the first standalone device is a standalone personal device comprising the plurality of sensors and devices for capturing details and medical information of the patient.

15. The system of claim 11 wherein the first standalone device is a standalone mobile device configured for connect to a plurality of portable sensors and devices for capturing details and medical information of the patient.

16. The system of claim 11, wherein the plurality of sensors and devises comprise: an SPO₂ pulsioximeter, a spirometer, a finger print scanner, a cash receiving port, a blood sample receiving port, a urine sample receiving port, a payment accepting port, a receipt providing port, a glucometer sensor, a camera and retina scanner on the front portion of the standalone device, a voice scanner, and the like.

17. The system of claim 11, wherein the second standalone device comprises a computer device with a designated software program to enable a medical practitioner to check patient record, prescribe medicine, and the line.

18. The system of claim 11, wherein the prescribed medication is collected from a medicine dispensing port connected to a medicine storage in the first standalone device, or delivered using a manned or unmanned vehicle, or delivered using drones.

19. The system of claim 1, wherein the first standalone device comprises a database to house the details and medical information of the patient, wherein the first standalone device is not directly connected to the server, and the information is communicated to the server using a plugin device

20. A non-transitory computer-readable storage medium with an executable program stored thereon, wherein the program instructs one or more processors to perform the following steps:
providing a first standalone device and creating a designated software program in the first standalone device, wherein the first standalone device is configured for interacting with a patient and capturing details and medical information of the patient, wherein the first standalone device comprises a plurality of sensors and devices for capturing details and medical information of the patient;
providing a server in communication with the first standalone device, wherein the first standalone device is configured to communicate the medical information of the patient to the server, wherein the server stores the medical information of the patient;
providing a second standalone device, and creating a designated software program in the second standalone device, configured for interacting with a medical practitioner;
the server in communication with the second standalone device, wherein the server is configured to communicate the medical information of the patient to the second standalone device;
wherein a diagnosis is communicated by the second standalone device to the first standalone device via the server;
wherein the first standalone device is configured to provide the patient with a prescription or a medication.
